(19) United States
(12) Patent Application Publication
(10) Pub. No.: US 2014/0134253 A1
(43) Pub. Date: May 15, 2014

(54) DRY POWDER FOSFOMYCIN/TOBRAMYCIN FORMULATION FOR INHALATION

(71) Applicants: Johnny Lai, Seattle, WA (US); Josiah Brown, Seattle, WA (US); Kevin W. Stapleton, San Carlos, CA (US)

(72) Inventors: Johnny Lai, Seattle, WA (US); Josiah Brown, Seattle, WA (US); Kevin W. Stapleton, San Carlos, CA (US)

(73) Assignee: Gilead Sciences, Inc., Foster City, CA (US)

(21) Appl. No.: 14/115,916

(22) PCT Filed: May 6, 2012
(86) PCT No.: PCT/US2012/036260

§ 371 Date: Jan. 28, 2014

Related U.S. Application Data
(60) Provisional application No. 61/483,452, filed on May 6, 2011.

(51) Int. Cl. A61K 9/00; A61K 31/7036; A61K 31/665

(52) U.S. Cl. CPC A61K 9/0073; A61K 31/665; A61K 31/7036
USPC 424/489; 514/40

(57) ABSTRACT

The present invention provides an inhaled dry powder formulation containing a combination of fosfomycin salt and tobramycin-leucine compound particles. The use of such formulation for the treatment of patients who have Chronic Obstructive Pulmonary Disease (COPD) and who are experiencing or at risk of experiencing acute exacerbation, as well as patients who have other bacterial infections of the respiratory tract, particularly the lower respiratory tract, and methods for treating the same are also provided.

### FIELD OF THE INVENTION

The present invention relates to an inhaled dry powder formulation containing a combination of fosfomycin and tobramycin, the use of such formulation for the treatment of patients who have Chronic Obstructive Pulmonary Disease (COPD) and who are experiencing or at risk of experiencing acute exacerbation, as well as patients who have other bacterial infections of the respiratory tract, particularly the lower respiratory tract, and methods for treating the same.

### BACKGROUND OF THE INVENTION

A widely accepted therapy for treating respiratory infections caused by Gram-negative bacteria involves intravenous administration of a single antibiotic or combinations of antibiotics. Gibson et al., 2003 Am. J. Respir. Crit. Care. Med. 168(8):918-951; Ramsey, 1996 N. Engl. J. Med. 335(3):179-188. However, this method of treatment has several significant limitations including: (1) narrow spectrum of activity of existing antibiotics, (2) insufficient concentrations of antibiotic reaching the respiratory tract to ensure rapid onset and high rates of bacterial killing, and (3) development of adverse side effects due to high systemic concentrations of drug.

Aerosol administration of antibiotics (Conway, 2005 Chronic Repir. Dis. 2:35-41; O'Riordan, 2000 Respir. Care 45(7):836-845) addresses several of the limitations of parenteral administration (Flume and Klepser, 2002 Pharmacotherapy 22(3 Pt 2):71S-79S; Kuhn, 2001 Chest. 120:94 S-98S). It enables topical delivery of high concentrations of drug to the endobronchial spaces and reduces side effects by lowering systemic exposure to antibiotic. However, patients having chronic respiratory conditions, such as chronic obstructive pulmonary disease, cystic fibrosis and bronchiestasis, may receive prolonged and repeated antibiotic therapies over the entire duration of their adult lives. Gibson et al., 2003 Am. J. Respir. Crit. Care. Med. 168(8):918-951; Ramsey, 1996 N. Engl. J. Med. 335(3):179-188. Therefore, cumulative antibiotic toxicity and development of resistance remains a significant problem.

Chronic obstructive pulmonary disease (COPD), a smoking-related condition characterized by progressive and poorly reversible airflow obstruction and airway inflammation, is the fourth most common cause of death in developed countries. COPD is projected to be the third leading cause of global deaths in 2020 and is the only one of the four most common causes of death with an increasing mortality rate. In 2008 in the United States, there were an estimated 10 million patients diagnosed with chronic obstructive pulmonary disease (COPD). SDI COPD Claims Analysis, May 2009. Murray et al., 1997 Lancet 349: 1269-76. Approximately 7 million U.S. patients receive treatment for COPD. Mannino et al; The Epidemiology and Economics of COPD, Proc Am Thorac Soc 2007. In the US, direct COPD costs in 2002 were approximately $18.0 billion. Statistics from National Center for Health Statistics, National Health Interview Survey: Research for the 1995-2004 redesign, Hyattsville, Md.: U.S. Department of Health and Human Services, CDC, NCHS. Vital and Health Stat 2(126), 1999.

The clinical course of COPD is characterized by chronic disability, with intermittent, acute exacerbations which may be triggered by a variety of stimuli including exposure to pathogens, inhaled irritants (e.g., cigarette smoke), allergens, or pollutants. “Acute exacerbation” refers to worsening of a patient's COPD symptoms from his or her usual state that is beyond normal day-to-day variations, and is acute in onset. See, Rabe et al., 2007 Am J Res Crit Care Med, 176: 532-555. Acute exacerbations of COPD greatly affect the health and quality of life of patients with COPD. Bathoorn, E, Int J Chron Obstruct Pulmon Dis. 2008 3(2):217-229. Acute exacerbation of COPD is a key driver of the associated substantial socioeconomic costs of the disease. Approximately 73% ($13 billion) of direct COPD costs in 2002 were due to hospitalizations related to acute exacerbations of COPD. Investigators from the Burden of Obstructive Lung Disease (BOLD) Initiative have estimated the cumulative discounted cost of COPD care in the US to be $880 billion by 2020—an average of more than $44 billion per year over two decades. Lee et al., 2006 ATS Proceedings, 3:A598. Multiple studies have also shown that prior exacerbation is an independent risk factor for future hospitalization for COPD. Garcia-Aymerich et al., 2003, Thorax, 58:100-105. Hospitalization consumes roughly 70% of COPD healthcare expenditure in the US. McGhan et al., 2007, Chest, 132(6):1748-1755. Accordingly, for a new drug therapy to significantly reduce the health and economic costs of COPD, it must address acute exacerbations of COPD.

It is clear that there is a continued need for an improved method of treatment for acute and chronic respiratory infections caused by Gram-negative and Gram-positive bacteria, particularly multidrug resistant bacteria, such as P. aeruginosa. This is particularly evident in patients having chronic respiratory conditions where current therapies are limited by problems with development of resistance and toxicity. Such method of treatment would preferably comprise inhalation of an aerosolized antibiotic composition that delivers a therapeutically effective amount of the active pharmaceutical ingredients directly to the endobronchial space of the airways or to the nasal passages. Such treatment would ideally be efficacious, reduce the frequency of drug resistance, and improve safety.

PCT Publication No. WO2005/110022 to Gilead Sciences, Inc. (formerly Corus Pharma) discloses a fosfomycin plus tobramycin combination formulation for delivery by aerosolization. The concentrated fosfomycin/tobramycin combination formulation containing an efficacious amount of fosfomycin and tobramycin is able to inhibit susceptible bacteria. Fosfomycin and tobramycin are formulated in solution such that when reconstituted, the pH is between 4.5 and 8.0 or as a dry powder. Also disclosed is a method for treatment of respiratory tract infections by a formulation delivered as an aerosol having mass medium aerodynamic diameter predominantly from 1 to 5 microns, produced by a jet or ultrasonic nebulizer (or equivalent) or dry powder inhaler.

It is an objective of this invention to provide a dispersible dry powder formulation of fosfomycin and tobramycin having superior aerosol and flow properties, chemical stability, patient tolerability and which would allow for efficient pulmonary drug delivery.

### SUMMARY OF THE INVENTION

As a first aspect, the invention provides a dry powder aerosol formulation comprising, consisting of, or consisting essentially of, a combination of: (i) fosfomycin particles comprising a fosfomycin salt; and (ii) tobramycin-leucine particles comprising a leucine compound and (a) tobramycin base and sulfuric acid or (b) tobramycin sulfate, wherein the formulation comprises from about 35% to about 80% by weight of fosfomycin acid, from about 5% to about 30% by weight of tobramycin base, and from about 0.5% to about 35% by weight of the leucine compound.

Fosfomycin salts useful in the formulations herein include fosfomycin disodium, fosfomycin trometamol and fosfomycin calcium.

Leucine compounds of use in the formulations herein include leucine, isoleucine, and N-acetyl leucine (NAL) in racemic form or as individual enantiomers such as D-leucine, L-leucine, D-isoleucine, L-isoleucine, DL-isoleucine, N-acetyl-L-leucine, N-acetyl-D-leucine, and the like. Also useful are dimers, trimers, teramers, and penatmers of leucine, including dileucine and trileucine, as well as modified forms of leucine in which one or more of the leucine atoms have been substituted with another atom or functional group and the dispersing effects of the modified compound are substantially the same as that of the unmodified amino acid or peptide. The formulations herein can comprise a single leucine compound or a combination of two or more leucine compounds. In one embodiment, the tobramycin-leucine particles comprise N-acetyl leucine, tobramycin base and sulfuric acid. In another embodiment, the tobramycin-leucine particles comprise N-acetyl leucine and tobramycin sulfate.

In another aspect, the invention provides a method of treating a bacterial infection in the respiratory tract, particularly the lower respiratory tract, of a human by administering by inhalation to said human an effective amount of the formulation of the first aspect. For example, in certain embodiments, the bacterial infection comprises an infection by a bacterium selected from the group consisting of Burkholderia cepacia, Citrobacter species, Escherichia coli, Enterobacter species, Fusobacterium species, Haemophilus influenzae, Klebsiella pneumoniae, Klebsiella oxytoca, Moraxella catarrhalis, Proteus mirabilis, Prevotella species, Pseudomonas aeruginosa, Serratia marcescens, Stenotrophomonas maltophilia, Alcaligenes xylosoxidans, methicillin-resistant Staphylococcus aureus, methicillin-sensitive Staphylococcus aureus, Streptococcus pneumoniae, and (β-hemolytic Streptococcus species. In certain preferred embodiments, the bacterial infection comprises an infection by a bacterium selected from the group consisting of P. aeruginosa, S. aureus, and methicillin-resistant S. aureus (MRSA).

In another aspect, the invention provides a method for treating a human with chronic obstructive pulmonary disease (COPD), particularly one who is experiencing or at risk of experiencing acute exacerbation of COPD, comprising administering by inhalation to said human an effective amount of the formulation of the first aspect. In certain embodiments, the method is selected from the group consisting of: a method for treating a human with COPD who is experiencing or at risk of experiencing acute exacerbation of COPD; a method for reducing the frequency, severity or duration of acute exacerbation of COPD in a human; a method for treating one or more symptoms of acute exacerbation of COPD in a human; a method for reducing the frequency, severity or duration of one or more symptoms of acute exacerbation of COPD in a human; and a method for treating pulmonary inflammation associated with COPD in a human.

In another aspect, the invention provides a method for treating cystic fibrosis in a human by administering by inhalation to said human an effective amount of the formulation of the first aspect.

In another aspect, the invention provides a method for treating bronchiectasis in a human by administering by inhalation to said human an effective amount of the formulation of the first aspect.

In another aspect, the invention provides the use of the formulation of the first aspect in the manufacture of a medicine suitable for administration by inhalation, for treating a bacterial infection in the respiratory tract, particularly the lower respiratory tract, of a human. For example, in certain embodiments, the bacterial infection comprises an infection by a bacterium selected from the group consisting of Burkholderia cepacia, Citrobacter species, Escherichia coli, Enterobacter species, Fusobacterium species, Haemophilus influenzae, Klebsiella pneumoniae, Klebsiella oxytoca, Moraxella catarrhalis, Proteus mirabilis, Prevotella species, Pseudomonas aeruginosa, Serratia marcescens, Stenotrophomonas maltophilia, Alcaligenes xylosoxidans, methicillin-resistant Staphylococcus aureus, methicillin-sensitive Staphylococcus aureus, Streptococcus pneumoniae, and β-hemolytic Streptococcus species. In certain preferred embodiments, the bacterial infection comprises an infection by a bacterium selected from the group consisting of P. aeruginosa, S. aureus, and methicillin-resistant S. aureus (MRSA).

In another aspect, the invention provides the use of the formulation of the first aspect in the manufacture of a medicine suitable for administration by inhalation, for treating a human with COPD, particularly one who is experiencing or at risk of experiencing acute exacerbation of COPD. In certain embodiments, the use is selected from the group consisting of: a use for treating a human with COPD who is experiencing or at risk of experiencing acute exacerbation of COPD; a use for reducing the frequency, severity or duration of acute exacerbation of COPD in a human; a use for treating one or more symptoms of acute exacerbation of COPD in a human; a use for reducing the frequency, severity or duration of one or more symptoms of acute exacerbation of COPD in a human; and a use for treating pulmonary inflammation associated with COPD in a human.

In another aspect, the invention provides the use of the formulation of the first aspect in the manufacture of a medicine suitable for administration by inhalation, for treating cystic fibrosis in a human.

In another aspect, the invention provides the use of the formulation of the first aspect in the manufacture of a medicine suitable for administration by inhalation, for treating bronchiectasis in a human.

### DETAILED DESCRIPTION OF THE INVENTION

As used herein:
“FTI” refers to an aerosol formulation of fosfomycin and tobramycin which is suitable for administration by inhalation.
“NAL” refers to N-acetyl-leucine.
“4:1 fosfomycin:tobramycin” and “4:1 Fos:Tob” are synonymous and mean a dry powder pharmaceutical formulation containing a 4:1 ratio by weight of fosfomycin acid to tobramycin base such that the amount of fosfomycin is four times the amount of tobramycin (by weight). The terms “7:3 fosfomycin:tobramycin” and “7:3 Fos:Tob” similarly refer to a dry powder pharmaceutical formulation containing a 7:3 ratio by weight of fosfomycin acid to tobramycin base and the terms “9:1 fosfomycin:tobramycin” and “9:1 Fos:Tob” similarly refer to a dry powder pharmaceutical formulation containing a 9:1 ratio by weight of fosfomycin acid to tobramycin base.
“MMAD” means mass median aerodynamic diameter.
“Predominantly” means including at least 70%, preferably at least 90%, of the specified particle size.
“COPD” refers to chronic obstructive pulmonary disease as defined by GOLD (see, Background) and is treated herein as inclusive of the same disease known by the alternative expressions “chronic obstructive respiratory disease” (CORD), “chronic obstructive airways diseases” (COAD), “chronic obstruction lung disease” (COLD), and “chronic airway limitation” (CAL).
“Acute exacerbation(s)” and “acute exacerbations in humans with COPD” are synonymous and refer to worsening of a patient's COPD symptoms from his or her usual state, that is beyond normal day-to-day variations, and is acute in onset.
“Acute exacerbations of chronic bronchitis in humans with COPD” refers to worsening of a COPD patient's chronic bronchitis symptoms from his or her usual state, that is beyond normal day-to-day variations and is acute in onset. Chronic bronchitis symptoms include dyspnea, excessive cough, sputum production, sputum purulence, change in color of sputum, chest tightness, reduced exercise tolerance, and fatigue
“Acute bacterial exacerbations of chronic bronchitis in patients with COPD” refers to a clinical diagnosis of presumptive bacterial infection superimposed on a chronic pulmonary condition. The term is defined by the FDA Center for Drug Evaluation and Research (CDER) in the Guidance for Industry on “Acute Bacterial Exacerbations of Chronic Bronchitis in Patients with COPD: Developing Antimicrobial Drugs for Treatment,” August 2008, Clinical Antimicrobial Division, Revision 1. According to the FDA Guidance, acute bacterial exacerbations of chronic bronchitis in patients with COPD may be described as bronchial inflammation associated with the isolation of pathogenic bacteria from sputum or bronchial lavage specimens. The role of bacteria is complicated in acute exacerbations as chronic bacterial colonization may be present in the airways of patients with COPD. Latent bacterial infection may also contribute to persistent inflammation.
“Frequent exacerbator” refers to a human who suffers from or is undergoing treatment for COPD and who experiences at least 2, and more typically 3 or more, acute exacerbations during a 12 month period.
“CF” means cystic fibrosis.
“FEV₁” refers to forced expiratory volume in 1 second and is a typical objective measure of a patient's respiratory condition.
“FEV₁/FVC” refers to FEV₁/forced vital capacity.
“Minimal inhibitory concentration (MIC)” means the lowest concentration of antibiotic (s) that prevents visible growth after incubation for 18-20 hours at 35° C.
“Minimal bactericidal concentration (MBC)” means the lowest concentration of antibiotic that results in ≧3 Log₁₀ of bacterial killing.
“Time-dependent killing” refers to an antibiotic in which the essential pharmacodynamic parameter is the time that drug concentrations remain above the MIC such that drug concentrations higher than the MIC do not kill bacteria any faster or to a greater extent.
“Concentration-dependent killing” refers to antibiotics in which the essential pharmacodynamic parameter is the drug concentration, such that the higher the drug concentration achieved, the greater the rate and extent of bacterial killing.
“Bacteriostatic” means the antibiotic acts by inhibiting bacterial growth.
“Bactericidal” means the antibiotics acts by killing bacteria.

### Formulation

The present invention relates generally to dispersible dry powder formulations of fosfomycin and tobramycin having superior aerosol and flow properties, chemical stability, patient tolerability and which allows for efficient pulmonary drug delivery.

As a second aspect, the invention provides a dry powder aerosol formulation comprising, consisting of, or consisting essentially of, a combination of: (i) fosfomycin particles comprising a fosfomycin salt; and (ii) tobramycin-leucine particles comprising a leucine compound and (a) tobramycin base and sulfuric acid or (b) tobramycin sulfate, wherein the formulation comprises from about 41% to about 66% by weight of fosfomycin acid, from about 7% to about 21% by weight of tobramycin base, and from about 0.8% to about 18% by weight of the leucine compound.

Another aspect of the invention provides formulations within the ranges provided, by weight, wherein the ratio of fosfomycin acid to tobramycin base is in a ration of from about 7 to 9 parts fosfomycin acid to from about 1 to 3 parts tobramycin base. Within these ranges is included the embodiments of about 7 parts fosfomycin acid and from about 1 to 3 parts tobramycin base; about 8 parts fosfomycin acid and from about 1 to 3 parts tobramycin base; and about 9 parts fosfomycin acid and from about 1 to 3 parts tobramycin base. Also included are embodiments of ratios of fosfomycin acid (fos) and tobramycin base (tob) comprising 7 fos:3 tob; 7 fos:2 tob; 7 fos:1 tob; 8 fos:3 tob; 8 fos:2 tob; 8 fos:1 tob; 9 fos:3 tob; 9 fos:2 tob; and 9 fos:1 tob.

The formulations of the present invention address a number of limitations associated with conventional dry powder formulations. For example, dry powder formulations for topical delivery to the lung by inhalation are generally formulated without excipient or carrier and instead including only the active ingredients in a dry powder form having a suitable particle size for inhalation. In the present case however, a formulation of only active ingredients would produce a pH in the lung which is outside of the tolerable range.

In addition, conventional dry powder formulations typically contain a mix of the active ingredient and a suitable powder base (carrier/diluent/excipient substance) such as mono-, di- or poly-saccharides (e.g., lactose or starch), wherein the particle size of the excipient is much greater than the active ingredient to aid the dispersion of the formulation in the inhaler. While lactose is typically the preferred excipient for conventional dry powder formulations, in the present case the use of lactose as an excipient in a FTI formulation would be problematic for several reasons. First, the active pharmaceutical ingredient dose load for a FTI formulation is typically <1 mg. When lactose is used as a carrier particle, the drug load of the resulting formulation is limited to the total surface area of the lactose particles and is typically from 0.05 to 18% by weight. To deliver a larger dose of active ingredient, as in a FTI formulation, the overall formulation mass required would be higher than what is typically considered possible to administer in a reasonable number of doses by inhalation. Second, lactose possesses nucleophilic groups, which groups would be expected to react with the epoxide ring of fosfomycin to produce a lactose-fosfomycin adduct reaction product.

In turn, this would be expected to reduce the antibiotic efficacy of the formulation by reducing the available amount of fosfomycin. Third, lactose typically utilized in inhalation products is the monohydrate form, which is the most stable form of lactose. However, the water in the lactose monohydrate would increase the hydrolysis of the epoxide ring of fosfomycin to produce a glycol impurity. This conversion of fosfomycin to a glycol impurity would also be expected to reduce the antibiotic efficacy of the formulation by the same effect as mentioned previously. Fourth, the water in the lactose monohydrate would increase the formation of a fosfomycin-tobramycin reaction product impurity via capillary action. All of these potential effects associated with the use of lactose in the formulation would shorten the shelf-life or stability of a potential FTI formulation commercial product. While there may be other issues associated with the use of lactose in a fosfomycin tobramycin dry powder formulation, the foregoing problems are indicative of the reasons why conventional dry powder technology and solutions are insufficient to overcome the obstacles in producing such a formulation comprising a combination of fosfomycin and tobramycin. Accordingly, in certain embodiments, the formulation of the present invention is free, or substantially free, of lactose.

The formulations of the invention are active against important respiratory pathogens common in patients suffering from chronic respiratory infections including those patients suffering from COPD, CF or bronchiectasis. Examples of these respiratory pathogens include but are not limited to Gram-negative bacteria, such as Burkholderia cepacia, Citrobacter species, Escherichia coli, Enterobacter species, Fusobacterium species, Haemophilus influenzae, Klebsiella pneumoniae, Klebsiella oxytoca, Moraxella catarrhalis, Proteus mirabilis, Prevotella species, Pseudomonas aeruginosa, Serratia marcescens, Stenotrophomonas maltophilia, Alcaligenes xylosoxidans, methicillin-resistant Staphylococcus aureus, methicillin-sensitive Staphylococcus aureus, Streptococcus pneumoniae, and β-hemolytic Streptococcus species. In certain more specific examples, the respiratory pathogens include but are not limited to P. aeruginosa (including multidrug resistant P. aeruginosa) S. aureus, H. influenzae, M. catarrhalis, and Enterobacteriaceae.

The formulations of the invention are rapidly bactericidal and have activity comparable to tobramycin. Moreover, FTI formulations have been shown to reduce the development of antibiotic resistance. Fosfomycin, the major component of FTI, has a very favorable safety profile when administered parenterally. Additionally, studies have suggested that fosfomycin may reduce aminoglycoside-induced nephrotoxicity. Since tobramycin base constitutes up to about 21% of FTI on a weight basis, the cumulative toxic effects due to tobramycin could also be reduced by use of FTI in place of tobramycin.

Tobramycin must traverse the cytoplasmic membrane prior to interacting with the ribosome and initiating lethal events. The major component of FTI, fosfomycin, is a phosphonic acid derivative that inhibits the first step of peptidoglycan biosynthesis in the bacterial cell wall by irreversibly binding to the enzyme phosphoenolpyruvate (UDP-N-acetylglucosamine enolpyruval-transferase). The minor component, tobramycin, prevents protein biosynthesis by causing translational errors and by inhibiting translocation. Based on these well characterized mechanisms of action, previous studies suggest the enhanced activities of FTI may be due to increased uptake of tobramycin. The precise identity of the protein(s) that facilitate the energy-dependent transport of tobramycin across the inner membrane are currently unknown.

### A. Components

1. Fosfomycin

Fosfomycin,

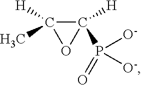

is a broad spectrum phosphonic acid antibiotic. Kahan, F. M., et al., 1974 Ann NY Acad Sci 253:364-386, and Woodruff et al., 1977 Chemother 23(1):1-22. Fosfomycin has bactericidal activity against Gram-negative bacteria including Citrobacter spp., Escherichia coli, Enterobacter spp., Klebsiella pneumoniae, P. aeruginosa, Salmonella spp., Shigella spp., and Serratia marcescens as well as Gram-positive bacteria including vancomycin resistant enterococci, methicillin-resistant S. aureus (MRSA), methicillin-sensitive S. aureus (MSSA), and S. pneumoniae. Greenwood et. al., 1992 Infection 20(4):S305-S309; Grimm, 1979 Infect 7(4):256-259; Marchese et. al., 2003 Int J Antimicrob Agents 22(2):53-59; and Schulin, 2002 J Antimicrob Chemother 49:403-406; and Perri et. al., 2002 Diagn Microbiol Infect Dis 42:269-271. Fosfomycin has the greatest activity against E. coli, Proteus spp., Salmonella spp., Shigella spp., and S. marcescens which are generally inhibited at fosfomycin concentrations ≦64 μg/mL (Forsgren and Walder, 1983 “Antimicrobial activity of fosfomycin in vitro” J Antimicrob Chemother 11(5):467-471). Fosfomycin is only moderately active against P. aeruginosa (Forsgren and Walder, supra), particularly when compared to tobramycin (Schulin, supra).

Fosfomycin is bactericidal but exhibits time-dependent killing against E. coli and S. aureus (Grif et al., 2001 J Antimicrob Chemother 48:209-217). The rate and degree of killing depends on the length of time fosfomycin is in contact with the target organism (Craig, 1998 Clin Infect Dis 26 (1):1-12; Mueller et al., 2004 Antimicrob Agents Chemother 48(2):369-377). Increasing the fosfomycin concentration will not produce a corresponding increase in the rate or degree of killing activity. This feature is significant because it is preferable to treat P. aeruginosa infections with antibiotics that exhibit bactericidal, concentration-dependent killing activity (Craig and Mueller et. al., supra).

Fosfomycin is widely distributed in various body tissues and fluids but does not significantly bind to plasma proteins. Consequently, fosfomycin is available to exert antibacterial effects if it reaches sufficient concentrations at the site of infection.

Fosfomycin inhibits the first step of peptidoglycan biosynthesis in the bacterial cell wall. FM Kahan, et al., 1974 Ann NY Acad Sci 235:364-386 and HB Woodruff, et al., 1977 Chemotherapy 23(Suppl 1):1-22. Fosfomycin has a high mutation frequency resulting in bacterial resistance in vitro. JL Martinez, et al., 2000 Antimicrob Agent Chemother 44:1771-1777 and Nilsson et al., 2003 Antimicrob Agents Chemother 47(9):2850-2858. When fosfomycin resistance occurs, it is typically due to a genetic mutation in one or both of the chromosomally encoded transport systems, and less commonly by modifying enzymes. Arca et al., 1997 J Antimicrob Chemother 40:393-399; and Nilsson et al., 2003, supra.

Fosfomycin is commercially available as fosfomycin disodium, fosfomycin trometamol and fosfomycin calcium. Both fosfomycin calcium and fosfomycin trometamol have been delivered orally while fosfomycin disodium is delivered intravenously. Only oral fosfomycin trometamol is approved in the USA for treating uncomplicated urinary tract infections. An aerosol formulation deliverable directly to the lungs is not yet commercially available.

The present invention may employ any form of fosfomycin, with the choice of the particular form of fosfomycin being well within the discretion of one skilled in the art. Fosfomycin calcium is used in one embodiment of the formulations, methods and therapeutic uses of the present invention. Formulations, methods and therapeutic uses utilizing fosfomycin disodium provides another embodiment and formulations, methods and therapeutic uses utilizing fosfomycin trometamol provides another embodiment.

2. Tobramycin

Tobramycin,

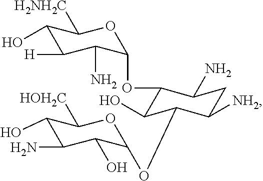

is an aminoglycoside antibiotic that is active against Gram-negative aerobic bacilli including P. aeruginosa, E. coli, Acinetobacter spp., Citrobacter spp., Enterobacter spp., K. pneumoniae, Proteus spp., Salmonella spp., S. marcescens, and Shigella spp (Vakulenko et al., 2003 Clin Microbiol Rev 16(3):430-450). In particular, tobramycin is highly active against P. aeruginosa. The tobramycin MICs of susceptible P. aeruginosa are typically less than 2 μg/mL (Shawar et al., 1999 Antimicrob Agents Chemother 43(12):2877-2880; Spencker et al., 2002 Clin Microbiol Infect 9:370-379; and Van Eldere, 2003 J Antimicrob Chemother 51:347-352). Most Gram-positive bacteria are resistant to tobramycin, with the exception of S. aureus and S. epidermidis (Vakulenko, et al., supra).

Tobramycin is rapidly bactericidal and acts by inhibiting bacterial protein synthesis. Tobramycin exhibits concentration-dependent killing. Increasing the tobramycin concentration increases both the rate and extent of bacterial killing. Therefore, to achieve therapeutic success, it is necessary to administer a large enough dose to produce a peak tobramycin level 5-10 times greater than the MIC of the target organism at the site of infection. It is preferable to treat P. aeruginosa infections with antibiotics that exhibit bactericidal, concentration-dependent killing activity (Ansorg et al., 1990 Chemother 36:222-229).

Tobramycin is usually administered to treat less serious Gram-negative bacterial infections (Vakulenko, et al., supra). However, it may be combined with other classes of antibiotics to treat severe infections of the urinary tract and tobramycin in combination with cell-wall inhibiting antibiotics has been used to treat respiratory infections, in particular those caused by P. aeruginosa in CF patients.

Tobramycin is poorly absorbed orally and must be administered parenterally. Tobramycin is available in both intravenous and aerosol formulations. After parenteral administration, tobramycin is primarily distributed within the extracellular fluid. Tobramycin is rapidly excreted by glomular filtration resulting in a plasma half-life of 1-2 hours. Tan et al., 2003 Am J Respir Crit. Care Med 167(6):819-823. Penetration of tobramycin into respiratory secretions is very poor and its activity is further reduced by binding to sputum (Kuhn, 2001 Chest 120:94 S-98S). Aerosol administration of tobramycin results in significantly higher sputum levels of ≧1000 μg/mL (Geller et al., 2002 Chest 122:219-226) compared with intravenous administration, but sputum binding remains a significant obstacle to antibiotic efficacy.

The respiratory tract of CF, bronciectasis and COPD patients are commonly obstructed with sputum. The effectiveness of several classes of antibiotics such as aminoglycosides and β-lactams is reduced due to poor penetration into sputum. Additionally, the activity of these antibiotics is further reduced by binding to sputum components. See, Hunt et al., 1995 Antimicrob Agents Chemother 39(1):34-39; Kuhn, 2001 Chest 120:94 S-98S; Ramphal et al., 1988 J Antimicrob Chemother 22:483-490; and Mendelman et al., 1985 Am Rev Respir Dis 132(4):761-765.

In addition, bacterial resistance to tobramycin has become increasingly prevalent and is due to repeated and prolonged antibiotic monotherapy. Conway et al., 2003 Am J Respir Med 2(4):321-332; Van Eldere, 2003 J Antimicrob Chemother 51:347-352; Mirakhur et al., 2003 J Cyst fibros 2(1):19-24; Pitt et al., 2005 Thorax 58(9):794-796; Schulin, 2002 J Antimicrob Chemother 49:403-406). For example, cystic fibrosis (CF) patients are colonized with P. aeruginosa strains which are largely resistant to tobramycin gentamicin, ceftazidinme, piperacillin, and ciprofloxacin. Thus, existing antibiotic therapies are becoming ineffective for treating P. aeruginosa infections because of drug resistance.

In the United States, the most widely used aerosolized antibiotic for treatment of CF patients is tobramycin inhalation solution (TIS), available under the TOBI® brand name from Novartis Pharmaceuticals Corporation, which has been shown to produce substantial improvements in pulmonary function and other clinical parameters in CF patients. TIS has been available in the United States for over 10 years, however, many clinicians are reluctant to use aerosolized tobramycin for chronic suppressive therapy fearing that long-term exposure could further promote resistance and diminish the effectiveness of intravenous aminoglycoside therapy. In order to reduce the risk of treatment-emergent resistance, TIS treatment in CF patients has been restricted to alternating courses of 28 days on drug followed by 28 days off drug. Aminoglycosides also have nephrotoxic and ototoxic effects that require routine monitoring of serum concentrations when administered intravenously. Aminoglycoside toxicity is cumulative and consequently, repeated administration by any route raises concern about total lifetime exposure to these agents.

As a consequence of the concern over toxicity, it would be beneficial to provide a dry powder formulation combining fosfomycin and tobramycin wherein tobramycin is present in minimal amounts which are effective when combined with fosfomycin, and which can be efficiently administered by dry powder inhalation.

Tobramycin is commercially available as a base or a salt (such as, tobramycin sulfate). Either form is suitable for use in the present invention, although, tobramycin sulfate is currently the preferred form of the compound for use in the formulation, methods and therapeutic uses of the present invention.

3. N-Acetyl-Leucine

N-Acetyl-leucine (NAL),

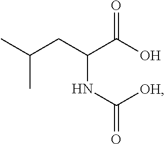

is commercially available as either the L or D isomer, N-acetyl-L-leucine or N-acetyl-D-leucine, respectively, or as the racemate, N-acetyl-DL-leucine. Either isomer, or a mixture thereof, is suitable for use in the present invention, although, N-acetyl-L-leucine is currently the preferred form of the compound for use in the formulation, methods and therapeutic uses of the present invention. Where a particular NAL isomer is used, it preferably has a high level of purity, e.g., greater than about 90% or 95% or 99%, or from about 90-100%, 95-100% or 99-100%. In certain preferred embodiments, for example, the NAL isomer used in the formulations of the invention comprises N-acetyl-L-leucine which is free or substantially free of the D form of NAL.

NAL can be used to adjust the pH of a dry powder aerosol formulation since NAL is acidic. The pH of the formulation is an important feature for aerosolized delivery. When the aerosol is either too acidic or too basic, it can cause bronchospasm and cough. Although the safe range of pH is relative and some patients may tolerate a mildly acidic aerosol, others, particularly those with chronic obstructive pulmonary disease, cystic fibrosis or other underlying diseases, are likely to experience bronchospasm upon inhalation of an acidic aerosol. Any aerosol with a pH of less than 4.5 typically induces bronchospasm. Aerosols with a pH between 4.5 and 5.5 will cause bronchospasm occasionally. Aerosol having pH greater than 8.0 should be avoided as the body tissues are unable to buffer alkaline aerosols. Thus, the ideal pH for an aerosol to be delivered by inhalation is between 4.5 and 8, and more preferably between 5.5 and 8.

Although simple amino acids and peptides have been used as excipients in pulmonary delivery, they exist as zwitterions with the free amine being protonated by the carboxylic acid. Since fosfomycin disodium and tobramycin base are basic with pHs of approximately 9.7, and 11 in solution, respectively, a formulation combining fosfomycin disodium and tobramycin base would be expected to be outside of the pH range to be tolerable for administration by inhalation to the pulmonary system. However, with the addition of NAL, the pH range in the lung upon administration of the dry powder formulation of the present invention is between about 7 to about 8, and preferably, from about 7.1 to about 7.7. Without the addition of NAL, a dry powder formulation combining fosfomycin disodium and tobramycin in the same molar ratios would be expected to produce a pH in the lung of about 8.2 (when the pH of tobramycin is adjusted with 2.5 molar equivalent of sulfuric acid) and a pH of about 9.1 (without pH adjustment with sulfuric acid).

4. Leucine

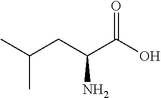

Leucine (Leu) is commercially available as either the L or D isomer, L-leucine or D-leucine, respectively, or as the racemate, DL-leucine. Either isomer, or a mixture thereof, is suitable for use in the present invention, although, L-leucine is the preferred form of the compound for use in some embodiments of the formulation, methods and therapeutic uses of the present invention. Where a particular isomer is used, it preferably has a high level of purity, e.g., greater than about 90% or 95% or 99%, or from about 90-100%, 95-100% or 99-100%. In certain preferred embodiments, for example, the L-isomer is used in the formulations of the invention which is free or substantially free of the D form of leucine.

5. Dileucine and Trileucine

Dileucine, also known as 2-((S)-2-amino-4-methylpentanamido)-4-methylpentanoic acid, Leucylleucine, L-Leucinyl-L-leucine, H-Leu-Leu-OH, L-leucyl-L-Leucine, and trileucine, also known as S)-2-((S)-2-((S)-2-amino-4-methylpentanamido)-4-methylpentanamido)-4-methylpentanoic acid, H-Leu-Leu-Leu-OH, Leu-Leu-Leu, and L-leucyl-L-leucyl-L-Leucine are also commercially available and useful in the formulations herein.

### B. Quantities and Ratios of Components

The dry powder aerosol formulation of the present invention generally comprises, consists of, or consists essentially of, a combination of (i) fosfomycin particles comprising a fosfomycin salt, and (ii) tobramycin-NAL particles comprising N-acetyl-leucine and (a) tobramycin base and sulfuric acid or (b) tobramycin sulfate.

A formulation of the invention may also contain other active components, or may be free or substantially free of other active components. In a specific embodiment, for example, a formulation of the invention comprises, consists of, or consists essentially of (i) fosfomycin particles comprising a fosfomycin salt, and (ii) tobramycin-NAL particles comprising N-acetyl-leucine and (a) tobramycin base and sulfuric acid or (b) tobramycin sulfate, and is free or substantially free of other components, such as lactose.

In certain embodiments, the formulation comprises from about 7 to about 9 parts by weight of fosfomycin acid and from about 1 to about 3 parts by weight of tobramycin base. In one embodiment, the formulation comprises about 4 parts by weight of fosfomycin acid and about 1 part by weight of tobramycin base. In another embodiment the formulation comprises about 7 parts by weight fosfomycin and about 3 parts by weight tobramycin base. In another embodiment the formulation comprises about 9 parts by weight fosfomycin and about 1 part by weight tobramycin base.

In some embodiments, tobramycin (namely, (i) tobramycin base and sulfuric acid or (ii) tobramycin sulfate) is spray-dried together with the leucine compound, such as NAL, to produce spray-dried tobramycin-leucine particles. Typically, in this embodiment, the tobramycin-leucine particles comprise from about 7% to about 40% by weight of the leucine compound. For example, in various embodiments, tobramycin-leucine particles, such as tobramycin-NAL particles, comprise about 7%, about 14%, about 22% or about 31% by weight of NAL. In one embodiment, tobramycin-NAL particles comprise about 14% by weight of NAL. With the addition of the leucine compound, such as NAL, the pH range in the lung upon administration of the dry powder formulation of the present invention is between about 7 to about 8, and preferably, from about 7.1 to about 7.7 (with 2.5 molar equivalence of sulfuric acid and 14 to 31% by weight of leucine compound, such as N-acetyl-leucine).

Typically, the specific amounts of fosfomycin acid and tobramycin base in the formulation, on a per dose basis will be in the range of from about 10 to 40 mg of fosfomycin acid and from about 2.5 to about 10 mg of tobramycin base.

Accordingly, one set of examples of a dry powder formulation wherein the ratio of fosfomycin acid to tobramycin base is about 4 parts by weight of fosfomycin acid to about 1 parts by weight of tobramycin base is a dry powder formulation comprising, with corresponding percentages for fosfomycin disodium: (1) about 56.2% fosfomycin acid (74.9% fosfomycin disodium), about 14.1% tobramycin base, about 7.4% sulfuric acid, and about 3.6% NAL; (2) about 54.8% fosfomycin acid (73.1% fosfomycin disodium), about 13.7% tobramycin base, about 7.2% sulfuric acid, and about 6.0% NAL; or (3) about 53.1% fosfomycin acid (70.8% fosfomycin disodium), about 13.3% tobramycin base, about 7.0% sulfuric acid, and about 9.0% NAL.

In another embodiment, the formulations herein comprise, by weight, from about 25-35% fosfomycin acid, from about 6-9% tobramycin base, and from about 1-3% leucine compound, such as leucine, NAL, dileucine, or trileucine.

In these formulation examples, the fosfomycin particles and tobramycin-leucine particles have a particle size suitable for inhalation (typically, from about 1 μm to about 10 μm, or from about 1 μm to about 5 μm).

Optional lubricants useful in the formulations herein include magnesium stearate, calcium stearate, sodium benzoate, sodium stearyl fumarate, and sodium stearyl lactylate. Lubricants may comprise up to 20% percent by weight of the present formulations. In one embodiment, one or more lubricants comprise from one to five percent of the present formulation, by weight.

Optionally, the compositions herein may also contain surfactants known in the art for use in dry powder pulmonary formulations. Non-limiting examples include phospholipids, such as dipalmitoylphosphatidylcholine (DPPC), other phosphatidylcholines, phosphatidylglycerols, ionic surfactants, such as sodium sulfosuccinates, non-ionic surfactants, polymers, and the like.

### C. Dosing

The aerosol formulations are presented in unit dosage form containing a predetermined amount of each of the active ingredients (e.g., fosfomycin and tobramycin) and the excipient NAL per unit dose. Preferred unit dosage formulations for the aerosol formulation are those containing an effective amount of the combination of fosfomycin and tobramycin, or an appropriate fraction thereof.

The amounts of fosfomycin and tobramycin contained in each unit dose, e.g., as described herein, may be optimized using conventional knowledge in the art based upon a number of factors, including the condition being treated, the route of administration, the bioavailability of the compounds, the species being treated, and the age, weight and condition of the patient. Unit dosage compositions typically contain a daily dose or a sub-dose or appropriate fraction thereof, of the active ingredients. Unit doses may be administered one or more times daily for the treatment of a particular condition.

The methods described herein are carried out by administering an effective amount of the dry powder formulation of fosfomycin and tobramycin by inhalation, to the human in need of treatment for a respiratory bacterial infection. The term “effective amount”, as used herein, is an amount of the combination of fosfomycin and tobramycin which is sufficient in the subject to which it is administered, to elicit the biological or medical response of a cell culture, tissue, system, that is being sought, for instance by a researcher or clinician. In one embodiment, the effective amount is the amount needed to provide a desired level of drug in the secretions and tissues of the airways and lungs, or alternatively, in the bloodstream of a subject to be treated, to give an anticipated physiological response or desired biological effect when such a composition is administered by inhalation. For example, in one embodiment, an effective amount of the combination is an amount sufficient in the subject to which it is administer to treat a bacterial infection in the respiratory tract of the human or to treat cystic fibrosis in a human, or to treat bronchiectasis is in a human.

An effective amount of the combination of fosfomycin and tobramycin in the dry powder formulation of the present invention will contain less of each component than would be required for a therapeutic effect if each component were delivered separately. For example the FDA approved dose for tobramycin inhalation solution is 300 mg of tobramycin 2 times per day. The dose of tobramycin powder for inhalation in clinical development is proposed to be 112 mg, 2 times per day.

A formulation of the present invention, in contrast, may contain from about 2.5 to about 10 mg of tobramycin per unit dose. The precise effective amount of the combination will depend on a number of factors including but not limited to the species, age and weight of the subject being treated, the precise condition requiring treatment and its severity, the bioavailability, potency, and other properties of the compounds being administered, the nature of the formulation, the route of administration, and the delivery device, and will ultimately be at the discretion of the attendant clinician.

Delivery of an effective amount of the combination of fosfomycin and tobramycin may entail delivery of a single dosage or multiple unit doses which may be delivered contemporaneously or separate in time over a designated period, such as 24 hours. Typically, the aerosol formulation will be administered four, three, or two times per day, or once per day (24 hours). In one embodiment, the aerosol formulation containing an effective amount of the combination will be administered two times per day (i.e., over a 24 hour period). In one particular embodiment, the aerosol formulation containing an effective amount of the combination will be administered twice per day (i.e., over a 24 hour period) for several consecutive days, particularly from 7 to 14 days.

The aerosol formulation according to the present invention is designed for administration by inhalation. Inhaled antibiotics offer advantages over intravenous therapy because relatively high drug concentrations can be delivered to the site of infection with minimal systemic absorption, thus reducing the risk of side effects associated with IV exposure.

The lung dose of the aerosol formulation will vary depending upon the selected dose of each component drug in the aerosol formulation and the efficiency of the delivery device. It is well established that the efficiency may vary among different dry powder inhalers. It is currently believed that a suitable lung dose of FTI for the methods and uses of the present invention will be from about 40 to about 10 mg fosfomycin acid and 10 to about 2.5 mg tobramycin per dose.

### D. Process for Preparing Dry Powder Formulation

The formulation of the present invention may be prepared as described below and using conventional methods in the art of pharmacy.

In one embodiment, the present invention provides a process for the preparation of a dry powder aerosol formulation comprising a combination of fosfomycin and tobramycin, wherein the process comprises:

(a) preparing fosfomycin particles comprising a fosfomycin salt;

(b) preparing tobramycin-leucine compound particles comprising the leucine compound and (i) tobramycin base and sulfuric acid or (ii) tobramycin sulfate; and

(c) blending a particulate mixture of the fosfomycin and tobramycin-leucine compound particles.

In one embodiment of the process above, a combination of two or more leucine compounds are used to prepare the tobramycin-leucine compound particles. In another embodiment NAL is used and in a further embodiment leucine is used.

In one embodiment, the tobramycin-leucine compound particles are prepared by first forming an aqueous solution of tobramycin base, and adjusting the pH of the solution with a suitable acid, particularly sulfuric acid, so that the solution pH is approximately 6.8. Alternatively, as one of skill in the art will appreciate, such a solution may be prepared using a commercially available tobramycin salt (such as tobramycin sulfate). The desired amount of leucine compound, such as NAL or leucine, is dissolved in ethanol. The two solutions are combined and the pH of the final solution is adjusted with a suitable acid, particularly sulfuric acid, so that 5 molar equivalents of protons are added. For example, with the use of sulfuric acid it will be 2.5 molar equivalents to obtain 5 molar equivalents of protons and the solution pH will be approximately 3.3. The final concentration is 2.5 to 5% (w/v). The solution is then spray-dried to produce spray-dried tobramycin-NAL particles. With the addition of the leucine compound, the pH range of the formulation in solution of the present invention is between about 7 to about 8, and preferably, from about 7.1 to about 7.7 (with 2.5 molar equivalents of sulfuric acid and 14-31% by weight of leucine compound). In different embodiments, tobramycin-leucine, tobramycin-NAL, tobramycin-dileucine, and tobramycin trileucine particles are prepared in this manner.

Typically, in conventional spray-dried dry powder formulations all the active pharmaceutical ingredient(s) and excipient(s) are solubilized and spray-dried together to produce one type of particle containing all components. However, due to the reactive nature of the epoxide ring in fosfomycin, spray-drying fosfomycin and tobramycin together produces large amounts of a fosfomycin-tobramycin reaction product impurity. In addition, with the fosfomycin and tobramycin molecules being in such proximity, the formation of such a reaction product impurity would be continuous and would shorten the shelf-life or stability of a potential FTI formulation commercial product. Accordingly, as described herein, in the formulations of the present invention, the fosfomycin particles are produced by micronization of fosfomycin, particularly fosfomycin disodium, and, separately, the tobramycin-NAL particles are produced by spray-drying a solution comprising N-acetyl-leucine and (i) tobramycin base and sulfuric acid or (ii) tobramycin sulfate. The fosfomycin and tobramycin-NAL particles are then blended to produce the dry powder formulation.

Spray-drying is a technique useful for the preparation of inhalable dry powder formulations. Typically, spray-drying involves spraying a fine mist of a solution containing the active pharmaceutical ingredient(s) (e.g., N-acetyl-leucine and (i) tobramycin base and sulfuric acid or (ii) tobramycin sulfate) into a drying chamber and collecting the resulting particles.

Spray-drying tobramycin with a leucine compound, such as leucine or N-acetyl-leucine, results in particles with superior aerosol and flow properties as compared to micronized tobramycin (produced by, e.g., jet milling) in the desired particle size range. Powder flowability is influenced by particle size, particle shape, surface texture, surface energy, chemical composition, moisture content, and other factors. The surface energy of particles is governed by mechanical interlocking, capillary forces, electrostatics, and van der Waals forces. The surface of spray-dried tobramycin-leucine compound particles, such as tobramycin-NAL particles, is smoother and more hydrophobic. This is due to the spray-drying particle formation process in which the hydrophobic leucine compound will tend to concentrate at the air-liquid interface. These smoother hydrophobic particle surfaces will reduce inter-particle mechanical interlocking and van der Waals forces. In addition, due to the smoother and more hydrophobic particle surfaces, there will be less potential unfavorable interactions between the two components (i.e., tobramycin and fosfomycin) than if they were both micronized. As a result, a combination of micronized fosfomycin particles and spray-dried tobramycin-leucine compound particles, such as tobramycin-NAL particles, would result in a better aerosolizing and flowing formulation than a combination of micronized fosfomycin particles and micronized tobramycin particles.

Micronization of fosfomycin disodium to the desired particle size range is required even though the resulting material exhibits poorer aerosol and flow properties as described above. Spray-drying fosfomycin disodium with an excipient (such as, leucine, isoleucine, and trileucine) that possesses a powerful nucleophile like an amine produces the corresponding reaction product during the spray-drying process and during subsequent stability studies. While spray-drying fosfomycin with N-acetyl-leucine does not produce any impurities during the spray-drying process, the resulting spray-dried fosfomycin disodium NAL product yields a reaction product with fosfomycin and the carboxylic acid functional group of N-acetyl-leucine during stability studies.

### E. Aerosol Properties and Delivery Devices

The formulation of the present invention is an inhalable dry powder pharmaceutical composition which is suitable for inhalation and delivery to the endobronchial space. Such composition is in the form of a dry powder aerosol formulation comprising particles for delivery using, for example, a dry powder inhaler (DPI).

Aerosols for administration of medicaments to the respiratory tract are typically polydisperse, that is they are comprised of particles of many different sizes. The particle size distribution is typically described by the Mass Median Aerodynamic Diameter (MMAD) and the Geometric Standard Deviation (GSD). For optimum drug delivery to the endobronchial space the MMAD is in the range from about 1 to about 10 μm and preferably from about 1 to about 5 μm, and the GSD is less than 3, and preferably less than about 2. Aerosols having an MMAD above 10 μm are generally too large to reach the lungs when inhaled. Aerosols with a GSD greater than about 3 are not preferred for lung delivery as they deliver a high percentage of the medicament to the oral cavity. To achieve these particle sizes in powder formulations, the particles of the active ingredient may be size reduced using conventional techniques such as micronization or spray-drying. The desired fraction may be separated out by air classification or sieving. Preferably, the particles will be crystalline.

Aerosol particle size distributions are determined using devices well known in the art. For example a multi-stage Anderson cascade or Next-Generation impactor or other suitable method such as those specifically cited within the US Pharmacopoeia Chapter 601 as characterizing devices for aerosols emitted from metered-dose and dry powder inhalers.

Non-limiting examples of dry powder inhalers include reservoir multi-dose inhalers, pre-metered multi-dose inhalers, capsule-based inhalers and single-dose disposable inhalers. A reservoir inhaler contains a large number of doses (e.g. 60) in one container. Prior to inhalation, the patient actuates the inhaler which causes the inhaler to meter one dose of medicament from the reservoir and prepare it for inhalation. Examples of reservoir DPIs include but are not limited to the Turbohaler® by AstraZeneca and the ClickHaler® by Vectura.

In a pre-metered multi-dose inhaler, each individual dose has been manufactured in a separate container, and actuation of the inhaler prior to inhalation causes a new dose of drug to be released from its container and prepared for inhalation. Examples of multidose DPI inhalers include but are not limited to Diskus® by GSK, Gyrohaler® by Vectura, and Prohaler® by Valois. During inhalation, the inspiratory flow of the patient accelerates the powder out of the device and into the oral cavity. For a capsule inhaler, the formulation is in a capsule and stored outside the inhaler. The patient puts a capsule in the inhaler, actuates the inhaler (punctures the capsule), then inhales. Examples include the Rotohaler™ (GlaxoSmithKline), Spinhaler™ (Novartis), HandiHaler™ (IB), TurboSpin™ (PH&T). With single-dose disposable inhalers, the patient actuates the inhaler to prepare it for inhalation, inhales, then disposes of the inhaler and packaging. Examples include the Twincer™ (U Groningen), OneDose™ (GFE), Manta Inhaler™ (Manta Devices).

Generally, dry powder inhalers utilize turbulent flow characteristics of the powder path to cause the excipient-drug aggregates to disperse, and the particles of active ingredient are deposited in the lungs. However, certain dry powder inhalers utilize a cyclone dispersion chamber to produce particles of the desired respirable size. In a cyclone dispersion chamber, the drug enters a coin shaped dispersion chamber tangentially so that the air path and drug move along the outer circular wall. As the drug formulation moves along this circular wall it bounces around and agglomerates are broken apart by impact forces. The air path spirals towards the center of the chamber exiting vertically. Particles that have small enough aerodynamic sizes can follow the air path and exit the chamber. In effect, the dispersion chamber works like a small jet mill. Depending on the specifics of the formulation, large lactose particles may be added to the formulation to aid in the dispersion through impact with the API particles. In other embodiments, however, the formulation will preferably be free or substantially free of lactose.

In preferred embodiments, the aerosol formulation is delivered as a dry powder using a dry powder inhaler wherein the particles emitted from the inhaler have an MMAD in the range of about 1 μm to about 5 μm and a GSD about less than 2.

Examples of suitable dry powder inhalers and dry powder dispersion devices for use in the delivery of compounds and compositions according to the present invention include but are not limited to those disclosed in U.S. Pat. Nos. 7,520,278; 7,322,354; 7,246,617; 7,231,920; 7,219,665; 7,207,330; 6,880,555; 5,522,385; 6,845,772; 6,637,431; 6,329,034; 5,458,135; 4,805,811; and U.S. Published Patent Application No. 2006/0237010.

In preferred embodiments, the dry powder inhaler is one that uses a cyclone dispersion chamber, illustrative examples of which include but are not limited to the Twincer™ inhaler and the Novilizer inhaler (See, e.g., U.S. Pat. Nos. 7,617,822 and 6,681,768, the contents of which are incorporated herein by reference).

In one embodiment, the pharmaceutical formulation according to the invention is a dry powder for inhalation which is formulated for delivery by a Diskus®-type device. The Diskus® device comprises an elongate strip formed from a base sheet having a plurality of recesses spaced along its length and a lid sheet hermetically but peelably sealed thereto to define a plurality of containers, each container having therein an inhalable formulation containing a predetermined amount of the formulation according to the present invention. Preferably, the strip is sufficiently flexible to be wound into a roll. The lid sheet and base sheet will preferably have leading end portions which are not sealed to one another and at least one of the leading end portions is constructed to be attached to a winding means. Also, preferably the hermetic seal between the base and lid sheets extends over their whole width. To prepare the dose for inhalation, the lid sheet may preferably be peeled from the base sheet in a longitudinal direction from a first end of the base sheet.

In one embodiment, the pharmaceutical formulation according to the invention is a dry powder for inhalation which is formulated for delivery using a single-dose disposable inhaler, and particularly the Twincer™ inhaler. The Twincer™ single-dose disposable inhaler appears to operate using a coin-shaped cyclone dispersion chamber referred to as an “air classifier.” See, U.S. Pat. No. 7,617,822 to Rijksuniversiteit Groningen. Papers published by the University of Groningen have stated that a 25 mg dose of pure micronized colistin sulfomethate could be effectively delivered as an inhalable dry powder utilizing this technology, including those by Westerman et al., J Cystic Fibrosis 6 (2007) 284-292; de Boer et al., Eur. J. Pharmaceutics and Biopharmaceutics 54 (2002) 17-24; and Le Brun et al., Eur. J. Pharmaceutics and Biopharmaceutics 54 (2002) 25-32.

The Twincer™ inhaler comprises a foil laminate blister with one or more recesses and a lid sheet hermetically but peelably sealed thereto to define a plurality of containers. Each container has a predetermined amount of the formulation of the present invention. The lid sheet will preferably have a leading end portion which is constructed to project from the body of the inhaler. The patient would operate the device and thereby administer the aerosol formulation by 1) removing the outer packaging overwrap, 2) pulling the foil tab to uncover the drug in the blister and 3) inhaling the drug from the blister.

It should be understood that in addition to the ingredients particularly mentioned above, the formulations of this invention may include other agents conventional in the art having regard to the type of formulation in question.

### Indications

### A. Chronic Obstructive Pulmonary Disease (COPD)

COPD is defined by the Global Initiative for Chronic Obstructive Lung Disease (GOLD) as “a disease state characterized by airflow limitation that is not fully reversible. The airflow limitation is usually both progressive and associated with an abnormal inflammatory response of the lungs to noxious particles or gases.” RA Pauwels et al., 2001 Am J Respir Crit. Care Med 163:1256-1276. Airflow limitation is the slowing of expiratory airflow as measured by spirometry, with a persistently low forced expiratory volume in 1 second (FEV₁). The FEV₁ percent predicted is used to divide patients into 4 grades of severity. The GOLD definition of airflow limitation is an FEV₁/FVC ratio of less than 70%. Id.

Previously, COPD was characterized by the classic Venn diagram depicting COPD at the intersection of three overlapping disease entities: chronic bronchitis, emphysema, and asthma. Chronic bronchitis is clinically defined as excessive cough and sputum production on most days for at least three months during at least two consecutive years. Emphysema is characterized by chronic dyspnea (shortness of breath) resulting from destruction of lung tissue and enlargement of air spaces, and expiratory flow limitation. Bronchiectasis is an abnormal stretching and enlarging of respiratory passages caused by a cycle of infection, inflammation and tissue damage in the airways. Asthma is an inflammatory disease of lung airways that makes the airways prone to constrict too much and too easily in response to stimuli. Asthma differs from COPD in that the loss of pulmonary function in asthma is reversible. The GOLD definition of COPD does not differentiate between chronic bronchitis and emphysema but does note that although asthma and COPD can coexist, the largely reversible airflow limitation in asthma merits different therapeutic approaches than the largely irreversible airflow limitation of COPD. Mannimo, Hospital Physician October 2001 22-31.

Common symptoms of COPD include dyspnea, sputum, coughing, upper airway symptoms such as colds and sore throats, wheezing, chest tightness, fatigue, fluid retention, and acute confusion. An acute exacerbation of COPD is typically a noticeable change from the COPD patient's baseline, typical or day-to-day condition. Thus, acute exacerbations may manifest as worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, acute confusion, and combinations of any two or more of these symptoms.

Recent data indicate that persistent bacterial airway infection develops at the earliest stages of COPD in roughly 30% of patients. Monso et al., 1999 European Respir J 13:338-42. This may be facilitated by smoke-related damage to innate lung defenses such as mucociliary clearance and the epithelial barrier. Curtis et al., 2007 PATS 4:512-521. Moreover, patients susceptible to frequent exacerbations appear to have significantly higher levels of inflammatory markers in induced sputum as well as latent bronchial infections. One study found exacerbation frequency to be significantly related to latent bronchial infection (p=0.023) while bacterial load in the stable state was found to be significantly correlated with sputum IL-8 levels (P=0.02). Patel et al., 2002, Thorax 57:759-764. Accordingly, it is currently believed that anti-infective drug therapy may reduce acute exacerbations in COPD in frequent exacerbators.

Common causes of acute exacerbations include inflammation, particularly chronic inflammation, infection, including chronic or persistent infection, pollution and allergens. Among the pathogenic triggers, 24% are believed to be viral, 30% bacterial and 25% both viral and bacterial. Papi 2006 Amer J Respir Crit Care Med 173:114-121. Viral pathogens associated with acute exacerbations in patients with COPD include rhinoviruses, influenza, parainfluenza, coronavirus, adenovirus, and respiratory syncytial virus.

Sethi and Murphy 2008 NEJM 359(22):2355-2365, reviewed the bacterial species typically responsible for acute exacerbations of COPD. According to their findings, Haemophilus influenzae (Gram-negative) occurs in 20-30% of exacerbations; Streptococcus pneumoniae (Gram-positive) and Moraxella catarrhalis (Gram-negative) each occur in 10-15% of exacerbations and P. aeruginosa (Gram-negative) occurs in 5-10% of exacerbations. Id. Chlamydophila pneumoniae (Gram-negative) and Mycoplasma pneumoniae contribute to 1-5% of the exacerbations. Id. Additionally, Legionella pneumoniae (Gram-negative) may be another etiologic agent. Id. Other bacterial species that may play a role (albeit less frequently) include Haemophilus haemolyticus (Gram-negative), Haemophilus parainfluenzae (Gram-negative), Enterobacter species (Gram-negative) and Staphylococus aureus (Gram-positive).

In August 2008, the FDA published an industry guide for program development and the design of clinical trials for antimicrobial drug products for the treatment of acute bacterial exacerbations of chronic bronchitis in patients with COPD (ABECB-COPD). FDA Center for Drug Evaluation and Research (CDER) in the Guidance for Industry on “Acute Bacterial Exacerbations of Chronic Bronchitis in Patients with COPD: Developing Antimicrobial Drugs for Treatment,” August 2008, Clinical Antimicrobial Division, Revision 1. According to that guidance, the pathogens most commonly associated with acute exacerbations in patients with COPD are S. pneumoniae, H. influenzae, and M. catarrhalis, and as such the goal of ABECB-COPD clinical trials should be to demonstrate an effect of antibacterial therapy on the clinical course of ABECB-COPD presumptively associated with these species.

Acute exacerbations, regardless of their trigger, are typically treated with increased bronchodilation, systemic corticosteroids and/or oral antibiotics. Current therapies including inhaled corticosteroids, long acting beta agonists and long acting muscarinic antagonists have shown a 20-25% decrease in exacerbations in long-term studies. An estimated 60-88% of patients that have exacerbations are treated with antibiotics. Adelphi COPD DSP VII 2008 and Adams et al., 2000 Chest 117:1345-1352. Unfortunately, there is no single antibiotic of choice for treatment of exacerbations in COPD and long-term effects are a concern particularly in the prevalence of antibiotic resistance.

Reducing the frequency, severity and duration of exacerbations is a key unmet need in the treatment of COPD patients. The overall effect of frequent acute exacerbations in patients with COPD contributes to more rapid reduction in quality of life, morbidity, mortality, and healthcare costs. It is currently believed that patients with COPD who are suffering from or susceptible to acute exacerbations, particularly moderate-to-severe COPD patients who are frequent exacerbators, will benefit from treatment with antibiotics, particularly in an antibiotic regimen involving intermittent course of short duration. This strategy targets both the bacterial pathogens that have latently infected lower airways—potentially reducing underlying airway inflammation and subsequent risk of acute exacerbations—as well as any new bacterial pathogens (or strains of pathogens) that may trigger an acute exacerbation.

Antibiotics may offer an additional advantage over anti-inflammatory agents, particularly inhaled corticosteroids, in the treatment of COPD, in that antibiotic therapy may target the upstream stimulus to the inflammatory cascade characteristic of COPD and thereby potentially avoid the pitfalls of redundant inflammatory pathways. Furthermore, antibiotics would not disable appropriate host-mediated immune response to pathogens. While inhaled corticosteroids have been shown (with and without long-acting beta₂ agonists) to reduce incidence of acute exacerbations, they are also associated with increased risk of pneumonia. See, Calverley et al., 2007, NEJM, 356:775-89.

### B. Respiratory Bacterial Infections

A wide variety of gram-negative bacteria, such as Burkholderia cepacia, Citrobacter species, Escherichia coli, Enterobacter species, Fusobacterium species, Haemophilus influenzae, Klebsiella pneumoniae, Klebsiella oxytoca, Moraxella catarrhalis, Proteus mirabilis, Prevotella species, Pseudomonas aeruginosa, Serratia marcescens, Stenotrophomonas maltophilia, and Alcaligenes xylosoxidans, and Gram-positive bacteria, such as methicillin-resistant Staphylococcus aureus, methicillin-sensitive Staphylococcus aureus, Streptococcus pneumoniae, and β-hemolytic Streptococcus species, cause severe pulmonary infections. Many of these bacteria are or become resistant to commonly used or specialty antibiotics and require treatment with new types of antibiotics. The pulmonary infections caused by gram-negative bacteria are particularly dangerous to patients who have decreased immunoprotective responses, such as, for example, cystic fibrosis patients, patients with bronchiectasis or those on mechanical ventilation (such as those with ventilator associated pneumonia or ventilator associated tracheobronchitis).

Therefore, the respiratory bacterial infections caused by organisms resistant to antibiotics continues to be a major problem, particularly in immunocompromised or hospitalized patients, as well as in patients assisted by mechanical ventilation, as described in Principles and Practice of Infectious Diseases, Eds. Mandel, G. L., Bennett, J. E., and Dolin, R., Churchill Livingstone Inc., New York, N.Y., (1995).

Currently accepted therapy for severe bacterial respiratory tract infections, particularly for treatment of pneumonia in patients with underlying illnesses, includes treatment with various intravenous antibacterial agents, often used in two or three way combination. Most of these agents are not suitable, available or FDA approved for either oral or aerosol dosing. In some cases the efficacious systemic intravenous or oral dose, if oral delivery is possible, requires doses which are borderline or outright toxic thus often preventing a use of perfectly good antibiotic for treatment of the pulmonary infections.

Thus it would be desirable to have available other modes of delivery routes of these antibiotics enabling a targeted delivery of smaller amounts of the antibiotic to endobronchial space of airways for treatment of these bacterial infections rather than administering the antibiotic systemically in large amounts.

Additionally, chronically ill patients are often affected with infections caused by bacteria which are largely resistant to commonly used antibiotics or, upon extended use of certain antibiotic, often develop strong resistance to such antibiotic. For example, chronic pulmonary colonization with Pseudomonas aeruginosa in patients with cystic fibrosis is a principal cause of their high mortality. When established, the chronic pulmonary infection is very difficult, if not impossible, to eradicate. More than 60% of cystic fibrosis patients are colonized with Pseudomonas aeruginosa bacterium strains which are largely resistant to regular and specialty antibiotics, such as piperacillin, ticarcillin, meropenem, netilmicin and only little sensitive to azlocillin, ciprofloxacin, timentin and ceftazidime. Many strains have also been shown to develop resistance to tobramycin and to colistin, if used continuously.

Often, after prolonged antibiotic therapy, a superinfection with organisms intrinsically resistant to oral, intravenous or inhaled antibiotics develops in patients with cystic fibrosis and other chronic pulmonary infections. The four most common drug resistant organisms are Burkholderia cepacia, Stenotrophomonas maltophilia, Alcaligenes xylosoxidans, and multidrug resistant Pseudomonas aeruginosa.

Cystic fibrosis patients infected with Burkholderia cepacia have an increased rate of mortality compared to those patients with Pseudomonas aeruginosa infections. In some cystic fibrosis patients, Burkholderia cepacia can cause a rapid fatality, as described, for example in Am. J. Respir. Crit. Care Med., 160: 5, 1572-7 (1999).

The high level of antibiotic resistance demonstrated by most strains of Burkholderia cepacia severely limits therapeutic options for its treatment (Clinics Chest Med., 19:473-86 (September 1998)). Furthermore, unlike Pseudomonas aeruginosa, Burkholderia cepacia can cause epidemic spread among cystic fibrosis patients and therefore any patient infected with Burkholderia cepacia is usually isolated from other patients. This causes both additional expenses connected with caring for these patients and may also be psychologically devastating to the patient. Furthermore, most lung transplant centers will not perform a lung transplant on patients infected with Burkholderia cepacia (Clinics Chest Med., 19:473-86 (September 1998)). Therefore, the Burkholderia cepacia infection is often viewed as a death sentence by patients with cystic fibrosis. Burkholderia cepacia is usually resistant to the parenteral delivery of various antibiotics with showing only 5% of isolates to be sensitive to such treatment (Antimicrob. Agents Chemother., 34: 3, 487-8 (March 1990)). Thus it would be advantageous to have available treatment for Burkholderia cepacia infections.

Other gram-negative bacteria intrinsically resistant to tobramycin can also complicate the care of a cystic fibrosis patient. These bacteria include Stenotrophomonas maltophilia and Alcaligenes xylosoxidans. Antibiotic therapy of these infections is usually also ineffective or leads to rapid emergence of drug resistance. Therefore, the successful treatment of all these infections requires that samples of these isolates are sent to a laboratory for complex antibiotic synergy determination of proper therapy for each individual patient (Ped. Pulmon., S17: 118-119 (1998)). It would, therefore, be also advantageous to provide a therapy for these rare but hard to treat bacterial infections.

Similarly, the development of P. aeruginosa infection with strains which are resistant to, that is which have a high minimal inhibitory concentration (MIC) to a majority of antibiotics, including tobramycin, predicts declining lung function and also may disqualify the patient from consideration for lung transplant (Clinics Chest Med., 19:535-554 (September 1998)).

Existing antibiotic treatments for Burkholderia cepacia, Stenotrophomonas maltophilia, Alcaligenes xylosoxidans, and multidrug resistant Pseudomonas aeruginosa pulmonary infections are either ineffective, or lead to rapid emergence of drug resistance. Accordingly, it is clear that there is a continuous need for an effective therapy for treatment of acute and chronic pulmonary bacterial infections, such as those caused by gram-negative bacteria and particularly those caused by Burkholderia cepacia, Stenotrophomonas maltophilia, Alcaligenes xylosoxidans, and multidrug resistant Pseudomonas aeruginosa lung infections. Such therapy would preferably comprise an inhalation of the aerosolized drug formulation delivering a therapeutically effective amount of the drug directly to the endobronchial space of airways to avoid systemic treatment and reduce the development of resistant infections.

### Methods of Treatment and Uses

Generally, the present invention provides methods of treating bacterial infections in the respiratory tract, particularly the lower respiratory tract, and other related conditions in humans using the inhalable formulations described herein. In particular, the present invention provides methods of treating bacterial infections in the respiratory tract, particularly the lower respiratory tract, of a human by administering to the lung endobronchial space by inhalation an effective amount of the formulation of the present invention.

In various embodiments, the bacterial infections may comprise an infection by a bacterium selected from the group consisting of Burkholderia cepacia, Citrobacter species, Escherichia coli, Enterobacter species, Fusobacterium species, Haemophilus influenzae, Klebsiella pneumoniae, Klebsiella oxytoca, Moraxella catarrhalis, Proteus mirabilis, Prevotella species, Pseudomonas aeruginosa, Serratia marcescens, Stenotrophomonas maltophilia, Alcaligenes xylosoxidans, methicillin-resistant Staphylococcus aureus, methicillin-sensitive Staphylococcus aureus, Streptococcus pneumoniae, and β-hemolytic Streptococcus species. In particular, infections by a bacterium selected from the group consisting of P. aeruginosa, S. aureus, and methicillin-resistant S. aureus (MRSA).

In certain embodiments, the present invention also provides methods for treating humans with COPD, cystic fibrosis and/or bronchiectasis, as well as methods for treating a bacterial infection in the respiratory tract, particularly the lower respiratory tract, of humans with COPD, cystic fibrosis or bronchiectasis. In certain embodiments the bacterial infection is ventilator associated pneumonia or ventilator associated tracheobronchitis.

“Treating” and “treatment”, as used herein refer to reversing, alleviating, inhibiting the progress of, or preventing the disorder or condition or one or more symptoms of the disorder or condition. For example, in particular embodiments of the present invention, “treating” refers to treating an acute exacerbation of COPD, reducing the frequency, duration or severity of an acute exacerbation of COPD, treating one or more symptoms of acute exacerbation of COPD, reducing the frequency, duration or severity of one or more symptoms of an acute exacerbation of COPD, preventing the incidence of acute exacerbation of COPD, preventing the incidence of one or more symptoms of acute exacerbation of COPD, treating pulmonary inflammation associated with COPD, or treating a bacterial infection in the respiratory tract, particularly the lower respiratory tract, in a human.

The reduction in frequency, duration or severity of a symptom of an acute exacerbation of COPD is relative to the frequency, duration or severity of an acute exacerbation or symptom in the same human not undergoing treatment according to the methods of the present invention. A reduction in frequency, duration or severity of acute exacerbation or one or more symptoms of acute exacerbation may be measured by clinical observation by an ordinarily skilled clinician with experience treating COPD patients or by subjective self evaluations by the patient undergoing treatment. Clinical observations by an ordinarily skilled clinician may include objective measures of lung function such as FEV₁ or FEV₁/FVC, as well as the frequency with which intervention is required to maintain the patient in his or her most stable condition, and the frequency of hospital admission and length of hospital stay required to maintain the patient in his or her most stable condition.

The symptoms of acute exacerbation include worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, acute confusion, and combinations of any two or more of these symptoms. Not all of the foregoing symptoms are required for a worsening of the COPD patient's condition to be identified as acute exacerbation. Acute exacerbations may manifest in the form of a subset of these symptoms. Accordingly, the inventors contemplate the practice of the inventive methods wherein only a subset of the foregoing symptoms of acute exacerbation are present.

In various embodiments, the acute exacerbation may be acute exacerbation of chronic bronchitis in a human with COPD or acute bacterial exacerbation of chronic bronchitis in a human with COPD.

As noted above, in one embodiment, the present invention provides a method of reducing pulmonary inflammation in a human with COPD. Reducing pulmonary inflammation according to the methods of the present invention may have the effect of reducing destruction of airway tissue as well as improving lung function and reducing the frequency, duration and severity of acute exacerbations (or symptoms thereof) in patients with COPD.

The present invention also provides, in other embodiments, the use a formulation of the present invention in the manufacture of a medicine suitable for administration to a human in the context of a therapeutic indication described hereinabove.

The methods and uses of the present invention all comprise the step of administering by inhalation to the human, an aerosol formulation containing an effective amount of a combination of fosfomycin and tobramycin.

### EXAMPLES

### Example 1

### Study Design for Evaluating Safety and Efficacy in Humans

The FDA Guidance: Acute Bacterial Exacerbations of Chronic Bronchitis in Patients with Chronic Obstructive Pulmonary Disease: Developing Antimicrobial Drugs for Treatment specifically addresses the development of antimicrobial drugs for the treatment of exacerbations in this indication. The clinical program for the prevention of acute exacerbations in COPD patients will consider any applicable clinical development requirements from the guidance, taking into consideration the guidance's objective of treatment over prevention of exacerbations.

Clinical studies in COPD patients with a history of recurrent acute exacerbations will focus on reducing the frequency, duration or severity of exacerbations and also evaluate changes in FEV₁, Quality of Life, and health care utilization. The clinical development path will follow trial designs similar to other studies currently being conducted for reduction of acute exacerbations.

The proposed Phase 2 study is a multicenter, randomized, double-blind, placebo-controlled study to evaluate the safety and efficacy of FTI in patients with moderate-to-severe COPD (i.e., baseline FEV₁≦70% predicted) who are >40 years old and have had a minimum of 2 acute exacerbations in the preceding 12 months. The Phase 2 study will include two FTI arms—1) Fos:Tob 40 mg:10 mg and 2) Fos:Tob 20 mg:5 mg; a tobramycin 10 mg arm and matching placebo. In all arms, drug will be administered by DPI twice a day for 7 days of every 28-day period, for a total of at least 6 months. Each arm of the study is expected to require about 150 patients.

The primary endpoint will be time to first acute exacerbation requiring treatment, with onset of acute exacerbation determined by the clinical investigator using a protocol-specified definition. The FDA draft guidance on COPD states that the definition should be “clinically meaningful” and include criteria such as worsening of dyspnea, increased sputum volume, increased purulence of sputum, worsening in symptoms requiring changes in treatment, or worsening of symptoms requiring urgent treatment or hospitalization. Key secondary endpoints will include number, severity and duration of acute exacerbation as assess by both type of treatment (e.g. oral antibiotics, hospitalization) and clinical assessment; time to second exacerbation; and change from baseline in FEV₁% predicted. The FDA Guidance noted above recommends the use of a patient reported outcome (PRO) primary endpoint. One potential PRO instrument, the Exacerbations from Pulmonary Disease Tool (EXACT-PRO), is currently being developed for evaluating clinical response in acute bacterial exacerbations of chronic bronchitis in patients with COPD. In addition to change from baseline in FEV₁% predicted, other safety endpoints will also be assessed, including change of MICs from screening to end of study.

Based upon the outcome of the Phase 2 study, the more efficacious dose of FTI (assuming comparable safety) would then be evaluated in two, 12-month Phase 3 studies, which would also include a tobramycin arm at the dose of tobramycin component of FTI in the Phase 3 trial, and a matching placebo arm. The primary endpoint of these studies would be number, severity and duration of acute exacerbations. The study would ideally be powered to demonstrate superior efficacy of FTI compared with tobramycin.

### Examples 2 and 3

Batches of the formulations having the target composition (% weight/weight) in 50 mg nominal doses, water content not included, were prepared using conventional spray drying techniques for the compositions listed below. The tobramycin-NAL particles were prepared through conventional spray drying techniques to a mass median aerodynamic diameter (MMAD) of from about 1 μm to about 5 μm. In Example 2, tobramycin particles were spray dryed with N-acetyl leucine (NAL) and in Example 3 spray dryed with N-acetyl leucine (NAL) and sulfuric acid. The fosfomycin disodium particles were jet milled to an MMAD of from about 1 μm to about 5 μm.

| Example | Tobramycin | Fosfomycin | Fosfomycin | Tobramycin | | Sulfuric |
|---|---|---|---|---|---|---|
| Number | Component | Disodium | Acid | Base | NAL | Acid |
| 2 | Spray dried | 40.5 mg | 30.5 mg | 7.5 mg | 2.0 mg | |
| | with NAL | 81% | 61% | 15% | 4.0% | |

| Example | Tobramycin | Fosfomycin | Fosfomycin | Tobramycin | | Sulfuric |
|---|---|---|---|---|---|---|
| Number | Component | Disodium | Acid | Base | NAL | Acid |
| 3 | Spray dried | 37.5 mg | 28.5 mg | 7 mg | 1.75 mg | 3.72 mg |
| | w/ NAL, H₂SO₄ | 72.6% | 57% | 14% | 3.5% | 7.5% |

The powders of the formulations of Examples 2 and 3 were kept below 10% relative humidity (RH) during processing, handling and storage. The pH of a sample of a powder equivalent of 10 mg fosfomycin acid and 2.5 mg tobramycin base of the formulation of Example 2 in 10 mL of water was about 9 and that of Example 3 was about 7.5.

### Examples 4-7

Additional examples of formulations can be prepared by the methods described herein and known in the art to prepare fosfomycin disodium and tobramycin-NAL formulations having the concentrations of ingredients per 50 mg unit shown in the table below.

| Example | Tobramycin | Fosfomycin | Fosfomycin | Tobramycin | | Sulfuric |
|---|---|---|---|---|---|---|
| Number | Component | Disodium | Acid | Base | NAL | Acid |
| 4 | Spray dried | 36.3 mg | 27.2 mg | 11.7 mg | 2.0 mg | |
| | with NAL | 72.6% | 54.4% | 23.4% | 4.0% | |
| 5 | Spray dried | 44.4 mg | 33.3 mg | 3.7 mg | 1.9 mg | |
| | with NAL | 88.8% | 66.6% | 7.4% | 3.8% | |
| 6 | Spray Dried w/ | 41.1 mg | 30.8 mg | 3.4 mg | 1.8 mg | 3.7 mg |
| | NAL, H₂SO₄ | 82.2% | 61.6% | 6.8% | 3.6% | 7.4% |
| 7 | Spray Dried w/ | 33.6 mg | 25.2 mg | 10.8 mg | 1.8 gm | 3.8 mg |
| | NAL, H₂SO₄ | 67.2% | 50.4% | 21.6% | 3.6% | 7.6% |

Additional formulations comprising fosfomycin calcium and tobramycin-NAL having the component concentrations below can be prepared by techniques described herein and known in the art.

| Example | Tobramycin | Fosfomycin | Fosfomycin | Tobramycin | | Sulfuric |
|---|---|---|---|---|---|---|
| Number | Component | Calcium | Acid | Base | NAL | Acid |
| 8 | Spray dried | 36.3 mg | 27.2 mg | 11.7 mg | 2.0 mg | |
| | with NAL | 72.6% | 54.3% | 23.4% | 4.0% | |
| 9 | Spray dried | 40.25 mg | 31 mg | 7.75 mg | 2 mg | |
| | with NAL | 80.5% | 62% | 15.5% | 4% | |
| 10 | Spray dried | 44.2 mg | 34.0 mg | 3.8 mg | 2 mg | |
| | with NAL | 88.4% | 68% | 7.6% | 4% | |
| 11 | Spray Dried w/ | 33.2 mg | 25.6 mg | 11.0 mg | 2 mg | 3.8 mg |
| | NAL, H₂SO₄ | 66.4% | 51.2% | 22.0% | 4% | 7.6% |
| 12 | Spray Dried w/ | 37.4 mg | 28.8 mg | 7.2 mg | 1.8 mg | 3.6 mg |
| | NAL, H₂SO₄ | 74.8% | 57.6% | 14.4% | 3.6% | 7.2% |
| 13 | Spray Dried w/ | 40.7 mg | 31.3 mg | 3.5 mg | 2 mg | 3.8 mg |
| | NAL, H₂SO₄ | 81.4% | 62.6% | 7% | 4% | 7.6% |

Further formulations comprising fosfomycin trometamol, also known as fosfomycin tromethamine, and tobramycin-leucine particles having the component concentrations below can be prepared by techniques described herein and known in the art.

| Example | Tobramycin | Fosfomycin | Fosfomycin | Tobramycin | |
|---|---|---|---|---|---|
| Number | Component | Trometamol | Acid | Base | Leu |
| 14 | Spray dried | 39.1 mg | 20.1 mg | 8.9 mg | 2.0 mg |
| | with Leu | 78.2% | 40.2% | 17.8% | 4.0% |
| 15 | Spray dried | 42.3 mg | 22.5 mg | 5.7 mg | 2 mg |
| | with Leu | 84.6% | 45% | 11.4% | 4% |
| 16 | Spray dried | 45.3 mg | 24.1 mg | 2.7 mg | 2 mg |
| | with Leu | 90.6% | 48.2% | 5.4% | 4% |

1. A dry powder aerosol formulation comprising a combination of:
(i) fosfomycin particles comprising a fosfomycin salt, and
(ii) tobramycin-leucine particles comprising a leucine compound and (a) tobramycin base and sulfuric acid or (b) tobramycin sulfate,
wherein the formulation comprises from about 35% to about 80% by weight of fosfomycin acid, from about 5 to about 30% by weight of tobramycin base, and from about 0.5% to about 35% by weight of the leucine compound.

2. A dry powder aerosol formulation according to claim 1 comprising a combination of:
(i) fosfomycin particles comprising a fosfomycin salt; and
(ii) tobramycin-leucine particles comprising a leucine compound and (a) tobramycin base and sulfuric acid or (b) tobramycin sulfate,
wherein the formulation comprises from about 41% to about 66% by weight of fosfomycin acid, from about 7% to about 21% by weight of tobramycin base, and from about 0.8% to about 18% by weight of leucine compound.

3. The formulation according to claim 1 wherein said formulation comprises about 4 parts by weight of fosfomycin acid to about 1 parts by weight of tobramycin base and comprises from about 65% to about 80% by weight of fosfomycin acid, from about 12% to about 15% by weight of tobramycin base, and from about 2% to about 13% by weight of leucine compound.

4. The formulation according to claim 1 wherein the ratio of fosfomycin acid to tobramycin base, by weight, is 7:3, 4:1, or 9:1.

5. The formulation according to claim 1, wherein said tobramycin-leucine compound particles are prepared by spray-drying a solution comprising the leucine compound and (i) tobramycin base and sulfuric acid or (ii) tobramycin sulfate.

6. (canceled)

7. The formulation according to claim 1, wherein said fosfomycin and tobramycin-leucine compound particles have a mass median aerodynamic diameter (MMAD) of from about 1 μm to about 5 μm.

8. The formulation according to claim 1, wherein said leucine compound is selected from the group of leucine, N-acetyl leucine, dileucine, and trileucine.

9. (canceled)

10. The formulation according to claim 8, wherein said N-acetyl-leucine is N-acetyl-L-leucine.

11. The formulation according to claim 10, wherein said tobramycin-NAL particles comprise from about 7% to about 40% by weight of N-acetyl-leucine.

12. The formulation according to claim 11, wherein said tobramycin-NAL particles comprise about 7%, about 14%, about 22% or about 31% by weight of N-acetyl-leucine.

13. The formulation according to claim 1, wherein said formulation has a pH from about 7 to about 8 in solution.

14. The formulation according to claim 1, wherein the fosfomycin salt is selected from the group of fosfomycin disodium, fosfomycin calcium, and fosfomycin trometamol.

15. A method of treating a bacterial infection in the respiratory tract of a human by administering by inhalation to said human an effective amount of the formulation according to claim 1.

16. The method according to claim 15, wherein the bacterial infection comprises an infection by a bacterium selected from the group consisting of Burkholderia cepacia, Citrobacter species, Escherichia coli, Enterobacter species, Fusobacterium species, Haemophilus influenzae, Kiebsiella pneumoniae, Kiebsiella oxytoca, Moraxella catarrhalis Proteus mirabilis, Prevotella species, Pseudomonas aeruginosa, Serratia marcescens, Stenotrophomonas maltophilia, Alcaligenes xylosoxidans, methicillin-resistant Staphylococcus aureus, methicillin-sensitive Staphylococcus aureus, Streptococcus pneumoniae, and β-hemolytic Streptococcus species.

17. (canceled)

18. A method for treating a human with chronic obstructive pulmonary disease (COPD) comprising administering by inhalation to said human an effective amount of the formulation according to claim 1.

19. The method according to claim 18, wherein the method is selected from the group consisting of:
a method for treating a human with COPD who is experiencing or at risk of experiencing acute exacerbation of COPD;
a method for reducing the frequency, severity or duration of acute exacerbation of COPD in a human;
a method for treating one or more symptoms of acute exacerbation of COPD in a human;
a method for reducing the frequency, severity or duration of one or more symptoms of acute exacerbation of COPD in a human; and
a method for treating pulmonary inflammation associated with COPD in a human.

20. The method according to claim 19, wherein said acute exacerbation of COPD is manifested by one or more symptoms, or said one or more symptoms of acute exacerbation of COPD is, selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion.

21. The method according to claim 18, wherein said acute exacerbation is acute exacerbation of chronic bronchitis in a human with COPD or acute bacterial exacerbation of chronic bronchitis in a human with COPD.

22. A method for treating cystic fibrosis in a human by administering by inhalation to said human an effective amount of the formulation according to claim 1.

23-32. (canceled)
